(19) United States
(12) Patent Application Publication
(10) Pub. No.: US 2003/0235607 A1
(43) Pub. Date: Dec. 25, 2003

(54) Compositions and methods for inhibiting fungal growth and transference

(76) Inventor: Carol J. Buck, Princeton, NJ (US)

Correspondence Address: KAPLAN & GILMAN , L.L.P., 900NROUTE 9 NORTH, WOODBRIDGE, NJ, 07095, US

(21) Appl. No.: 10/406,428

(22) Filed: Apr. 3, 2003

Related U.S. Application Data
(63) Continuation-in-part of application No. 10/309,510, filed on Dec. 4, 2002.
Application No. 10/309,510 is a division of application No. 09/514,049, filed on Feb. 25, 2000, now Pat. No. 6,517,822.
Application No. 09/514,049 is a continuation-in-part of application No. 09/023,449, filed on Feb. 13, 1998, now abandoned.

(51) Int. Cl. A61K 31/19; A61K 9/70

(52) U.S. Cl. 424/443; 514/557

(57) ABSTRACT

Provided are a composition, article of manufacture and method for preventing and inhibiting fungal growth and transference, particularly compositions containing at least one alkanoic acid and methods of using same on or in substrates known to harbor fungi such as T. rubrum and T. mentagrophytes.

This application is a continuation-in-part of U.S. Ser. No. 10/309,510, filed Dec. 4, 2002, which is a divisional of U.S. Ser. No. 09/514,049, filed Feb. 25, 2000, now U.S. Pat. No. 6,517,822, which is continuation-in-part of U.S. Ser. No. 09/023,449, filed Feb. 13, 1998 and claims the benefit of the priority date thereof.

### FIELD OF THE INVENTION

This invention relates to compositions and methods for inhibiting fungal growth and transference, particularly compositions containing at least one alkanoic acid and methods of using same on or in substrates known to harbor fungi such as T. rubrum and T. mentagrophytes.

### BACKGROUND OF THE INVENTION

### Onychomycosis

Causes

Onychomycosis (OM) is the medical term for a fungal infection of the nail plate. OM accounts for one-third of all fungal skin infections and one-half of all nail disease. The primary dermatophytes (fungi that infect hair, skin, and nails) that cause onychomycosis are trichophyton rubrum and trichophyton mentagrophytes. T. rubrum and T. mentagrophytes are responsible for at least 80% of OM infections in temperate zones. Trichophyton rubrum (T. rubrum ) accounts for 80% of all dermatophyte nail infections and 46% of all nail infections. A less common type of onychomycosis is caused by yeast (candida albicans or candida parapsilosis). The incidence of yeast-related OM ranges from 5.4% to 6.3% and is predominantly found on fingernails, rather than toenails. A third, less common type, paronychia infections, are caused by bacteria such as staphylococcus, streptococcus, and pseudomonas. OM affects toenails, usually the hallux (large nail), much more frequently than fingernails. The pathogenesis of OM largely depends on the clinical subtype. The fungus generally spreads from plantar skin and invades the underside of the nail via the hyponychium or the distal lateral nail bed. Infection of the nail is often a result of tinea pedis infection between the toes. Once under the nail the fungus breaks down keratin into a digestible form of the protein called keratin debris. Keratin debris builds up under the nail causing increased thickness and the characteristic yellow-brownish color.

Acrylic nail use on fingernails is another cause of OM. Pockets of moisture develop between the acrylic material and the nail plate in which fungi can survive, creating over time the same conditions apparent in infected toenails. Recently, the wearing of acrylic nails has been banned for all medical practitioners because of their high fungal and bacteria content.

Incidence and Treatment

Incidence of OM in the US is estimated between 5% and 30% of the adult population with most cases going unreported. It is rare in children. Risk factors for OM include diabetes, family history, increasing age, poor health, prior trauma, warm climate, presence of athlete's foot fungus, participation in fitness activities, immuno-suppression (e.g., HIV, drug-induced), communal bathing, and occlusive footwear. There is usually no pain until the infection is advanced and the thickness of the nail causes pressure on underlying nerves and when pressed up against shoes, causing ambulatory impairment and discomfort.

The most commonly employed treatments for OM are terbinafine (Lamisil®) (administered orally or topically), itraconazole (Sporanox®) (orally) and ciclopirox 8% (Penlac® Nail Lacquer). These work gradually over several months to reduce or eliminate the infection. In some cases, adverse side effects of the oral medications occur in the liver, requiring all patients to monitor liver function and enzyme levels, pre and post treatment. Over the counter topicals are also commercially available, but contain active ingredients that are effective only for killing fungi on the skin surrounding the nail or nail surface infection, known as superficial OM.

Fungi is widespread in the environment, including shoes, rugs, tiled floors, gymnasiums, playing fields, etc., making exposure to the fungus inevitable. The current regimens for preventing fungal infection of the feet are maintaining clean, dry feet; a strict regimen of washing the feet with soap and water; wearing shower shoes in public areas; and changing shoes, socks, or hosiery daily. Use of drying talcum powder and synthetic fiber socks that draw moisture away are also recommended methods of fungal infection prevention and control. None of these procedures, however, are guaranteed to prevent infection. Since the fungus in invisible to the naked eye, there is no way to determine if shoes and surfaces are uncontaminated with dermatophytes capable of migrating under the nail.

U.S. Pat. No. 5,489,329 to Chappell discloses a wall-coating composition including potassium sorbate, cellulose gum and acetic acid. However, the active fungicidal ingredient is potassium sorbate and acetic acid is merely employed as a pH adjuster.

U.S. Pat. No. 6,413,425 discloses a combination of boric acid and acetic acid for use in a method for the treatment of infections of organs such as skin and vagina. This patent does not disclose a composition useful in preventing the growth and regrowth of the disease-causing dermatophytes T. rubrum and T. Mentagrophytes, in footwear and on surfaces where people walk barefoot.

There is a need, therefore, for a product that will maintain a fungi-free environment for the foot, including the insides of shoes including leather and canvas shoes and tile surfaces where persons walk barefoot in the course of bathing, especially in environments such as locker rooms, saunas and steam rooms. This product optimally is administered topically to surfaces or impregnated into the material of footwear or surfaces making contact with bare feet.

### SUMMARY OF THE INVENTION

The invention described herein provides compositions and methods for preventing the transference of fungi from the environment to the toenail and fingernail. The present invention provides a composition for the inhibition of T. rubrum and T. mentagrophytes in environments wherein contact of these fungi to human nail tissue will invariably result in onychomycosis (OM). While treatments exist for OM, unless the fungi that cause OM are eliminated from areas contacted by the OM sufferer's affected tissue, it is unlikely the OM will be eliminated. T. rubrum and T. mentagrophytes harbored in places such as shoes and locker rooms will opportunistically reinfect the OM sufferer each and every time the fungus-containing item is contacted. Therefore there is a need for a novel composition and method to eliminate and prevent the growth and regrowth of disease-causing dermatophytes, e.g., T. rubrum and T. Mentagrophytes, in footwear and on surfaces where people walk barefoot. The present invention provides a composition and method that safeguards against fungal re-invasion and provides a barrier to athlete's foot and nail fungus by killing microscopic fungi in shoes and on floor surfaces, thereby maintaining a fungi-free environment.

A method is also provided for the prevention of recurrence of OM wherein embodiments of the present invention are used in combination with NONYX® gel.

### OBJECTS OF THE INVENTION

It is therefore an object of a preferred embodiment of the present invention to provide a novel alkanoic acid composition having a fungicidal effect on T. rubrum and T. mentagrophytes.

It is a further object of a preferred embodiment of the present invention to provide a novel article comprising a carrier impregnated with a fungicidal alkanoic acid composition.

It is yet a further object of a preferred embodiment of the present invention to provide a novel method for applying an alkanoic acid to a substrate to inhibit fungal growth and transference from substrate to mammalian nails.

It is still a further object of a preferred embodiment of the present invention to provide a novel footwear insert comprising a suitable carrier impregnated with a fungicidal alkanoic acid composition.

It is yet another object of a preferred embodiment of the present invention to provide a composition added to a medicament to prevent growth of T. rubrum and T. mentagrophytes in said medicament.

### DETAILED DESCRIPTION OF THE PREFERRED EMBODIMENTS

The invention relates to compositions, carriers containing said compositions and methods for safely inhibiting the growth, regrowth and transference of the fungi T. rubrum and T. mentagrophytes. The inventive composition in one embodiment comprises a solution containing alkanoic acid and a diluent. The composition is safe and does not present a risk of irritating or burning the skin.

Advantageously, the composition contains greater than about 2.5% (w/w) alkanoic acid. With regard to the concentration of the alkanoic acid, optimum results are achieved when the composition contains in the range of about 5.5%-10% (w/w) of the alkanoic acid. Compositions having greater than about 10% (w/w) alkanoic acid are effective however do not provide the maximum safety benefits of the invention. When less than 30% (w/w) alkanoic acid is used, and more preferably when less than 20% (w/w) acetic acid is used, the composition is still effective in preventing growth, regrowth and transference of fungi. Thus, applicant considers compositions having from about 5.5% (w/w) to about 20% (w/w) alkanoic acid to be preferred, and compositions having from about 5.5% (w/w) to about 9.75% (w/w) alkanoic acid to be even more preferred. In one embodiment, the composition is used for safely inhibiting the growth, regrowth and transference of the fungi T. rubrum and T. mentagrophytes on surfaces in locations such as bathrooms, locker rooms, steam rooms and the like.

As used herein, the term “alkanoic acid” refers to carboxylic acids with alkane, alkene, or similar substituents. The preferred alkanoic acid is acetic acid, CH₃COOH. Other suitable alkanoic acids include but are not limited to methanoic, ethanoic, 2-methylbutanoic, propanoic, 2-methylpropanoic, 2,2 dimethylpropanoic, decanoic, octanoic, 2-hexenoic, heptanoic, 6-methylheptanoic, 3-ethylpentanoic, 3-chloropentanoic, 2-hydroxypropanoic, 2-chloro-4-hydroxyhexanoic, hexanedioic, octadecanoic, 4-oxopentanoic, and 6-hydroxy-4-oxonanoic acids.

The term “diluent” as used herein refers to substances that may be used to dilute the alkanoic acids. Water is the preferred diluent. At higher concentrations the pH may be as low as 1.5 while at lower concentrations the pH may be as high as 4.0. At the preferred aqueous dilution of 9.8%, the pH is 2.5. The invention teaches that a dilute solution of a weak, non-sulfur containing alkanoic acid (e.g., acetic acid) is effective to inhibit the growth, regrowth and transference of the fungi T. rubrum and T. mentagrophytes. The materials to be protected from such types of fungal contact can be, for example, hard, smooth surfaces, such as tile or decking, or porous, rough surfaces such as canvas or leather.

It will be appreciated that auxiliary ingredients may be added to the compositions to mask the scent of the acid and/or perform other functions such as substrate cleansing and/or conditioning. Also, gelling agents such as xanthan gum or other known gelling agents may be used to create a desired consistency or viscosity to the composition. Advantageously, such auxiliary components comprise less than 50% of the composition, more preferably less than 30%, and even more preferably less than 20/% of the compositions. The auxiliary components added to the compositions may include but are not limited to leather, floor and cloth conditioners and cleansing agents, polishes, bleaches, gel or foam polymers, cellular urethanes, silicone, Shea butter, lanolin, mineral oil, petrolatum, plant oils, paraffin, beeswax, sodium laureth sulfate, sodium lauryl sulfate, glycerin, steric acid, aloe, urea, ceto stearyl alcohol, perfuming masks such as menthol, wintergreen oil, floral and other fragrances, artificial colorants, and other enhancers.

In one embodiment the composition contains fragrance in the range of from about from 0.05% to 3.5%, preferably 0.25% to 1.5% and most preferably about 0.35% by weight.

The composition may also include between about 0.10% to about 2% of an emulsifier, surfactant or wetting agent such as but not limited to TWEEN®. In a preferred embodiment the composition contains about 0.25% by weight of an emulsifier, surfactant or wetting agent.

The composition may be dispensed in the form of an aerosol spray, liquid spray, gel or cream, or incorporated into cleaning agents such as tile cleaners, laundry detergents, floor cleaners, shoe and boot polishes, leather cleaners and conditioners, canvas shoe cleaners and whiteners, bathroom disinfectants, water purifiers for pools, spas, saunas and hot tubs, fabric treatments for foot apparel such as socks, sprays for athletic shoes, work shoes, ski boots and the like. The composition can be employed in conjunction with known mammalian antifungal agents. The composition may also be employed to impregnate suitable materials such as but not limited to inserts of canvas, leather, foam, polymer, gel or cellular urethane for footwear, or for pretreatment of materials such as but not limited to canvas, leather and other suitable materials used in the manufacture of footwear. According to yet another embodiment the composition may be added to a medicament such as but not limited to Penlac® Nail Lacquer to prevent growth of T. rubrum and T. mentagrophytes in said medicament.

In a most preferred embodiment the composition comprises a formulation of 89.40% deionized water, 9.75% acetic acid, 0.40% fragrance, 0.25% emulsifying agent, and 0.20% xanthan gum.

The composition can be employed in conjunction with other compositions and methods of treating OM such as but not limited to those disclosed in U.S. Pat. No. 6,231,840, incorporated herein by reference.

Method of Applying the Compositions to Surfaces.

An exemplary method of using the compositions of the present invention utilizes a 9.8% solution of acetic acid in deionized water, fragrance, emulsifying agent, and gum. The composition is applied to surfaces via a spray apparatus such as a bottle, pump or aerosol sprayer. Alternatively the composition may be applied using a sponge, mop, towels or the like.

Another exemplary method of using the compositions of the present invention utilizes a 9.8% solution of acetic acid in deionized water, fragrance, emulsifying agent, and gum. The composition is applied to natural or synthetic fabric, paper or foam carriers in the form of “wipes” and the “wipes” are applied to surfaces via a manual wiping operation. In a most preferred embodiment a plurality of carriers containing the composition in the form of “wipes” are provided in a container permitting convenient spot treatment of surfaces.

Another exemplary method of using the compositions of the present invention utilizes a 20% solution of acetic acid in deionized water, surfactant, and fragrance in concentrated liquid form for mixing with commercial cleaning products, particularly those used to sanitize bathrooms, pools, showers, health clubs, schools, gyms, weigh-lifting facilities, pools, spas, steam rooms, etc. An alternative method of using the composition is to provide it at the preferred concentration of 9.8% in large, industrial tanks equipped with commercial hoses to efficiently spray a large floor surface such as a pool or locker room area.

The following examples and experiments will serve to further typify the nature of the invention, but should not be construed as a limitation on the scope thereof, which is defined solely by the appended claims.

### EXAMPLE 1

| Ingredients | Weight % |
|---|---|
| Acetic Acid | 9.00 |
| Deionized water | 90.15 |
| Fragrance | 0.40 |
| Emulsifier | 0.25 |
| XANTURAL ® | 0.20 |
| gum powder (thickening agent) | 100% |

The ingredients are combined in the form of a liquid, thickened slightly with a gelling agent, such as xanthan gum.

According to one method of application, the formulation is applied via a spraying device such as but not limited to a pump or bottle sprayer to a shower floor, public dressing room floor, locker room or the like before entering stall. The floor may be wet or dry. Preferably the formulation is applied holding the sprayer about 12 inches above the floor and lightly spraying the area. The composition may optionally be removed from the surface after a suitable interval of time, usually at least ten seconds, such as by rinsing. Preferably the formulation should be permitted to work for at least 30 seconds before an individual enters the treated area or removing shoes.

According to another embodiment, the formulation is applied by spraying device to the interior surfaces of footwear once weekly. The footwear is allowed to dry before wearing. Footwear is preferably resprayed within ten days, more preferably once a week, to prevent the reinfection of shoe material and continue to protect feet from OM-causing fungus. Occasionally-worn footwear should be sprayed with the formulation 20 minutes before wearing.

According to yet another embodiment the composition is added to a medicament such as but not limited to Penlac® Nail Lacquer to prevent growth of T. rubrum and T. mentagrophytes in said medicament.

### EXAMPLE 2

| Ingredients | Weight % |
|---|---|
| 12% acetic acid | 75.00 |
| Tap water | 24.73 |
| XANTURAL ® gum powder | 0.20 |
| Winter Green Oil | 0.07 |
| | 100 |

This example was prepared in a 500gram batch by mixing the ingredients in a Pyrex mixing bowl using a Braun mixer. Viscosity can be adjusted by varying the amount of gum powder. Greater or lesser viscosity may be desired depending on the manner in which the formulation is to be applied.

In one embodiment this formulation is employed in a natural or synthetic paper, fabric or foam carrier in the form of a “wipe”. A plurality of such wipes are provided in a container from which the wipes can be dispensed, preferably one at a time. In another embodiment, such wipes can be adapted to be fixed to the end of a handle on a substantially flat mounting surface to be employed in a mop or broom-like fashion on surfaces to be treated.

In one embodiment the formulation of Example 1 is employed in a footwear insert. The insert comprises a natural or synthetic paper, fabric or foam carrier is impregnated with the formulation. The insert is placed in footwear prior to wearing and removed before wearing said footwear or optionally retained in the footwear after the insert has dried sufficiently to permit comfortable wearing of the footwear with the insert. Alternatively, the inserts would also be manufactured in the form of shoe liners, consisting of canvas, leather, foam, polymer, gel or cellular urethane and packaged in sealed pouches as replaceable shoe liners.

### Experiment 1

Antimicrobial activity of a formulation prepared in accordance with the invention was determined versus a commercially available terbinafine-containing (1%) product by determining the Minimal Inhibitory Concentration (MIC) and Minimum Lethal Concentration (MLC) of each against Trychophyton rubrum (T. rubrum ). The methods used in this study were designed to obtain basic information about the relative antimicrobial activity of the samples tested at different concentrations.

M02-2429.01=9.75% acetic acid/water/gel

M02-2429.02=1% terbinafine product

M02-2429.03=Placebo Gel

Culture Preparation:

The spores of a well-grown Sabouraud Dextrose Agar (SDA) plate of the fungal Trychophyton rubrum ATCC # 28188 were harvested using 5 mL of sterile saline. The spores were centrifuged and suspended in sterile saline. This suspension was used as the inoculum.

Samples Preparation:

A set of eight different dilutions of each sample was prepared using Sabouraud Dextrose Broth (SDB) as follows:

1) 2.0 grams sample+8.0 mL media=1:5 or 20%

2) 2.0 mL of #1+8.0 mL media=1:25 or 4%

3) 2.0 mL of#2+8.0 mL media=1:125 or 0.8%

4) 2.0 mL of #3+8.0 mL media=1:625 or 0.16%

5) 2.0 mL of #4+8.0 mL media=1:3125 or 0.032%

6) 2.0 mL of #5+8.0 mL media=1:15625 or 0.0064%

7) 2.0 mL of #6+8.0 mL media=1:78125 or 0.00128%

8) 2.0 mL of #7+8.0 mL media=1:390625 or 0.000256%

MIC/MLC Test:

Culture vortexed and 0.1 ml added to all the tubes containing the diluted test materials, vortex to mix. Incubated all the tubes at 24±2° C. for 5-7 days, or until evidence of growth observed in the control tubes. Tubes examined for evidence of growth i.e. turbidity. If evidence of growth is observed, the specific concentration of the test material is considered to have no effect on the growth of the fungi, however if no evidence of growth is observed, the test material is either fungistatic or fungicidal at that concentration. From the concentrations that showed no growth, transfer 0.1 mL to a new SDB tube, and incubated as above. If no evidence of growth is seen, the test material concentration under test is considered fungicidal. If evidence of growth is seen, the test material concentration under test is considered fungistatic. Positive and negative controls were employed.

Results:

| | Dilutions | | | | | | | |
|---|---|---|---|---|---|---|---|---|
| Sample | | | | | | | | 1: |
| # | 1:5 | 1:25 | 1:125 | 1:625 | 1:3125 | 1:15625 | 1:78125 | 390625 |

MIC Results

| | | | | | | | | |
|---|---|---|---|---|---|---|---|---|
| M02- | (−) | (−) | (−) | (+) | (+) | (+) | (+) | (+) |
| 2429.01 | | | | | | | | |
| T. | | | | | | | | |
| rubrum | | | | | | | | |
| M02- | (+) | (+) | (+) | (+) | (+) | (+) | (+) | (+) |
| 2429.02 | | | | | | | | |
| T. | | | | | | | | |
| rubrum | | | | | | | | |
| M02- | (+) | (+) | (+) | (+) | (+) | (+) | (+) | (+) |
| 2429.03 | | | | | | | | |
| T. | | | | | | | | |
| rubrum | | | | | | | | |

MLC Results

| | | | | | | | | |
|---|---|---|---|---|---|---|---|---|
| M02- | (−) | (−) | (−) | NA | NA | NA | NA | NA |
| 2429.01 | | | | | | | | |
| T. | | | | | | | | |
| rubrum | | | | | | | | |

(+) = visible growth
(−) = no visible growth

Conclusion:

The product composition of the 9.75% aqueous acetic acid gel was effective, giving an MIC and MLC result of antifungal activity against T. rubrum at 1:125 dilution, while both terbinafine 1% cream and the xanthan gum gel (water, xanthan gum and fragrance) had no effect against T. rubrum in this study.

### Experiment 2

The antifungal activity was determined for the test samples against Trychophyton Mentagrophytes (T. Mentagrophytes), on canvas, tile, and leather.

M03-0317.01=Concentration A=9.75% acetic acid and water (spray)

M03-0317.02=Concentration B=5.5% acetic acid and water (spray)

M03-0317.03=Concentration C=2.5% acetic acid and water (spray)

The spores of a well-grown plate of the fungal Trychophyton mentagrophytes ATCC # 9533 were harvested using 5 mL of sterile saline. The spores were centrifuged and suspended in sterile saline. This suspension used as inoculum.

The canvas, tile, and leather pieces were cut into one inch by one inch squares. Sabaroud Dextrose Agar (SDA) plates were poured and allowed to solidify. The materials were placed on the SDA plates. 0.1 mL of the inoculum was placed on each material provided. After inoculation, two spritzes of each appropriate spray was applied to the T. mentagrophytes and material. Plates were performed in duplicate for each material and corresponding spray concentration.

Control plates were performed simultaneously; the positive control consisted of two SDA plates inoculated with T. mentagrophytes only, as well as additional positive controls to show that the materials can support the growth of T. mentagrophytes. Negative controls were plates of SDA with no inoculum or materials. Plates were incubated at 20-25° C., for 10 days. Plates were observed for visible differences and recorded.

Results are listed below in comparison with each spray concentration and with each material sampled:

M03-0317.01+leather=no growth on the leather or in close proximity to the leather

M03-0317.01+tile=no growth on tile or in close proximity to the tile

M03-0317.01+canvas=no growth on canvas or in close proximity to the canvas

M03-0317.02+leather=no growth on leather, small zone of inhibition

M03-0317.02+tile=no growth on tile, small zone of inhibition

M03-0317.02+canvas=no growth on canvas, large zone of inhibition

M03-0317.03+leather=growth on leather and surrounding agar

M03-0317.03+tile=growth on tile and surrounding agar

M03-0317.03+canvas=growth on canvas and surrounding agar

Conclusion:

M03-0317.01 and M03-0317.02 concentrations worked most effectively against the fungal T. mentagrophytes on all three of the test materials.

### Experiment 3

The antifungal activity was determined for the test samples against Trychophyton rubrum on canvas, tile, and leather.

M02-2697.01 =Concentration A =9.75% acetic acid and water (spray)

M02-2697.02 =Concentration B =5.5% acetic acid and water (spray)

M02-2697.03 =Concentration C =2.5% acetic acid and water (spray)

The spores of a well-grown plate of the fungal Trychophyton rubrum ATCC #28188 were harvested using 5 mL of sterile saline. The spores were centrifuged and suspended in sterile saline. This suspension used as inoculum. The canvas, tile, and leather, supplied were cut into one inch by one inch squares. Sabaroud Dextrose Agar (SDA) plates were poured and allowed to solidify. The materials were placed on the SDA plates. 0.1 mL of the inoculum was placed on each material provided. After inoculation, two spritzes of each appropriate spray was applied to the T. rubrum and material. Plates are performed in duplicate for each material and corresponding spray concentration. Control plates were performed simultaneously; the positive control consisted of two SDA plates inoculated with T. rubrum , as well as positive controls to show that the materials can support the growth of T. rubrum . Negative controls were plates of SDA with no inoculum or materials. Plates were incubated at 20-25° C., for 10 days. Plates were observed for visible differences and recorded below.

Results are listed below in comparison with each spray concentration and with each material sampled:

M03-2697.01+leather=no growth on the leather or in close proximity to the leather

M03-2697.01+tile=no growth on tile, small zone of inhibition surrounding tile

M03-2697.01+canvas=no growth on canvas or agar

M03-2697.02+leather=no growth on leather, large zone of inhibition.

M03-2697.02 +tile =no growth on tile, small zone of inhibition in close proximity to tile

M03-2697.02+canvas=no growth on canvas, no growth in close proximity to canvas

M03-2697.03+leather=growth on leather and surrounding agar

M03-2697.03+tile=growth on tile and surrounding agar

M03-2697.03+canvas=no growth on canvas, growth on surrounding agar

Conclusion:

All three concentrations worked best on the canvas material, second best on the leather material, and least productive on the tile. 9.75% aqueous acetic acid was the concentration that had the best antifungal activity on all three of the test materials sampled.

### Experiment 4

The antifungal activity of the test sample was determined by determining the minimal inhibitory concentration/the minimal lethal concentration against T. mentagrophytes.

Test Materials:

M03-0486.01=9.75% acetic acid/water/gel

Organism Panel:

The following ATCC organism constituted the challenge panel: Trychophyton mentagrophytes ATCC # 9533

Procedure:

A. Culture Preparation:

A primary culture was prepared of the above microorganism using Sabouraud Dextrose Agar (SDA) plates, harvesting the plates using 5 mL sterile saline. The spores were centrifuged and suspended in sterile saline. The suspension was used as the inoculum.

B. Samples Preparation:

A set of 8 different dilutions of each sample was prepared using SDB as follows:

1) 11.2 mL sample+8.8 mL SDB=5.50%

2) 10.0 mL of # 1+10.0 mL SDB=2.75%

3) 10.0 mL of # 2+10.0 mL SDB=1.38%

4) 10.0 mL of # 3+10.0 mL SDB=0.69%

5) 10.0 mL of # 4+10.0 mL SCB=0.34%

6) 10.0 mL of # 5+10.0 mL SDB=0.17%

7) 10.0 mL of # 6+10.0 mL SDB=0.09%

8) 10.0 mL of# 7+10.0 mL SDB=0.04%

MIC/MLC test was performed as set forth in Experiment 1.

Results:

| | Dilutions | | | | | | | |
|---|---|---|---|---|---|---|---|---|
| Sample # | 5.5% | 2.75% | 1.38% | 0.69% | 0.34% | 0.17% | 0.09% | 0.04% |

MIC Results

| | | | | | | | | |
|---|---|---|---|---|---|---|---|---|
| M03-0486.01 | (−) | (−) | (+) | (+) | (+) | (+) | (+) | (+) |
| T. Mentagrophytes | | | | | | | | |

MLC Results

| | | | | | | | | |
|---|---|---|---|---|---|---|---|---|
| M022429.01 | (−) | (−) | NA | NA | NA | NA | NA | NA |
| T. Mentagrophytes | | | | | | | | |

NA = not applicable
(+) = visible growth
(−) = no visible growth

Conclusion:

The product composition of the test material shows fungicidal activity at the concentration of 2.75% against the fungal T. mentagrophytes.

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof which do not depart from the spirit and scope of the invention.

What is claimed is:

1. A composition for the prevention of contagion by T. rubrum or T. mentagrophytes fungi comprising:
(i) greater than about 2.5% to about 30% by weight of at least one alkanoic acid and
(ii) an acceptable diluent.

2. The composition of claim 1 said diluent comprising water.

3. The composition of claim 1 said diluent comprising at least 50% water by weight.

4. The composition of claim 1 in which the alkanoic acid is selected from the group consisting of acetic, methanoic, ethanoic, 2-methylbutanoic, propanoic, 2-methylpropanoic, 2,2 dimethylpropanoic, decanoic, octanoic, 2-hexenoic, heptanoic, 6-methylheptanoic, 3-ethylpentanoic, 3-chloropentanoic, 2-hydroxypropanoic, 2-chloro-4-hydroxyhexanoic, hexanedioic, octadecanoic, 4-oxopentanoic, and 6-hydroxy-4-oxonanoic acids.

5. The composition of claim 1 having a pH value of between about 1.5 to about 4.0 effective for inhibiting growth and transference of fungi.

6. The composition according to claim 5 in which said fungi is selected from the group consisting of T. rubrum and T. mentagrophytes.

8. The composition according to claim 1 comprising less than about 10% by weight of an alkanoic acid.

9. The composition according to claim 1 comprising less than about 5.5% by weight of an alkanoic acid.

10. An article comprising at least one natural or synthetic carrier and the composition according to claim 1.

11. The article according to claim 1 in which said carrier is selected from the group consisting of paper, cloth, leather, gel, polymer, cellular urethane and foam.

12 The invention according to claim 11 comprising a plurality of said carriers and further comprising a vessel for containing said carriers and said composition.

13. A composition for the prevention of contagion by T. rubrum or T. mentagrophytes fungi comprising:
(i) greater than about 2.5% to about 30% by weight of at least one alkanoic acid;
(ii) an acceptable diluent and
(iii) at least one auxiliary component selected from a surfactant, emulsifier, wetting agent, thickener, fragrance, odor masker, gelling agent, deodorizer, skin conditioner, lanolin, aloe, surface conditioner, cleansing agent, polish, bleach, gel polymer, foam polymer, cellular urethane, silicone, Shea butter, mineral oil, petrolatum, plant oil, paraffin, beeswax, sodium laureth sulfate, sodium lauryl sulfate, glycerin, stearic acid, urea, ceto stearyl alcohol, and a colorant.

14. A method for preventing infection by fungi causing onychomycosis comprising the step of applying at least a composition comprising greater than about 2.5% to about 30% by weight of at least one alkanoic acid and an acceptable diluent to a surface.

15. The method according to claim 14 wherein said surface is a floor of a public area.

16. The method according to claim 14 comprising the further step of subsequently removing said composition from said surface.

17. The method according to claim 16 wherein said composition is permitted to remain on said surface at least 10 seconds prior to removal.

18. The method according to claim 16 said removal step comprising rinsing said surface.

19. The method according to claim 14 wherein said surface is at least a portion of footwear.

20. The method according to claim 14 wherein said surface is at least a portion of material to be used in the fabrication or construction of footwear, comprising the step of treating said material prior to fabrication of said footwear.

21. A treatment method for preventing the reinfection of mammalian tissue by fungi causing onychomycosis comprising the steps of applying at least a composition comprising greater than about 2.5% to about 30% by weight of at least one alkanoic acid and acceptable diluent to surfaces likely to be contacted by said tissue and applying directly to said tissue a topical composition for the treatment of nail fungal conditions.

22. The method according to claim 21 wherein said composition applied to said tissue is selected from the group consisting of terbinafine and itraconazole.

23. The method according to claim 21 wherein said composition applied to said tissue comprises at least one alkanoic acid in aqueous solution wherein the ratio of alkanoic acid to water is greater than about 1:20, such that the concentration of said alkanoic acid is greater than about 5% by weight.

24. A fungicidal composition effective against T. rubrum and T. mentagrophytes fungi comprising:
(i) greater than about 2.5% to about 30% by weight of at least one alkanoic acid and
(ii) an acceptable diluent.

25. The invention according to claim 24 further comprising a medicament for the treatment of onychomycosis.

26. A method for preventing growth of onychomycosis-causing fungi, in medicaments, comprising the steps of providing a medicament and adding to said medicament a composition comprising greater than about 2.5% to about 30% by weight of at least one alkanoic acid and an acceptable diluent.
